SEQUENCE LISTING (7 sequences)

SEQ ID NO: 1 - DNA, 1061 bp, Lactobacillus crispatus
agccatgcaccacctgtcttagcgtccccgaagggaactttgtatctctacaaatggcactagatgtcaagacctggtaaggttcttcgcgttgcttcgaattaaaccacatgctccaccgcttgtgcgggcccccgtcaattcctttgagtttcaaccttgcggtcgtactccccaggcggagtgcttaatgcgttagctgcagcactgagaggcggaaacctcccaacacttagcactcatcgtttacggcatggactaccagggtatctaatcctgttcgctacccatgctttcgagcctcagcgtcagttgcagaccagagagccgccttcgccactggtgttcttccatatatctacgcattccaccgctacacatggagttccactctcctcttctgcactcaagaaaaacagtttccgatgcagttcctcggttaagccgagggctttcacatcagacttattcttccgcctgcgctcgctttacgcccaataaatccggacaacgcttgccacctacgtattaccgcggctgctggcacgtagttagccgtgactttctggttgattaccgtcaaataaaggccagttactacctctatccttcttcaccaacaacagagctttacgatccgaaaaccttcttcactcacgcggcgttgctccatcagacttgcgtccattgtggaagattccctactgctgcctcccgtaggagtttgggccgtgtctcagtcccaatgtggccgatcagtctctcaactcggctatgcatcatcgccttggtaagcctttaccttaccaactagctaatgcaccgcggggccatcccatagcgacagcttacgccgccttttaaaagctgatcatgcgatctgctttcttatccggtattagcacctgtttccaagtggtatcccagactatggggcaggttccccacgtgttactcacccatccgccgctcgctttcctaacgtcattaccgaagtaaatctgttagttccgctcgctcgacttgcatgtattaggcacgccgccagcgttcgtcctgag

SEQ ID NO: 2 - DNA, 20 bp, Artificial Sequence
  Oligonucleotide
caacaggcatccctaagtct

SEQ ID NO: 3 - DNA, 20 bp, Artificial Sequence
  Oligonucleotide
caacgtcttcagaccacatc

SEQ ID NO: 4 - DNA, 20 bp, Artificial Sequence
  Oligonucleotide
tctgcctcatgtagatccaa

SEQ ID NO: 5 - DNA, 20 bp, Artificial Sequence
  Oligonucleotide
gctccatctttaagacccaa

SEQ ID NO: 6 - DNA, 20 bp, Artificial Sequence
  Oligonucleotide
aagtgttatgggcctagtcg

SEQ ID NO: 7 - DNA, 20 bp, Artificial Sequence
  Oligonucleotide
gccaccacttttgctttaat

(19) United States
(12) Patent Application Publication
(10) Pub. No.: US 2015/0190438 A1
(43) Pub. Date: Jul. 9, 2015

(54) NOVEL STRAIN OF LACTOBACILLUS CRISPATUS

(71) Applicant: S.P.M.D., Paris (FR)

(72) Inventor: Adrien Nivoliez, Yolet (FR)

(73) Assignee: S.P.M.D., Paris (FR)

(21) Appl. No.: 14/413,303

(22) PCT Filed: Jul. 9, 2013
(86) PCT No.: PCT/EP2013/064424

§ 371 Date: Jan. 7, 2015

(30) Foreign Application Priority Data
Jul. 9, 2012 (FR) 1256569

(51) Int. Cl. A61K 35/747; A61K 9/00; A61F 13/20; C12R 1/225

(52) U.S. Cl. CPC A61K 35/747; C12R 1/225; A61K 9/0036; A61F 13/2074

(57) ABSTRACT

The disclosure relates to an isolated strain of Lactobacillus crispatus, identified as IP174178 and deposited at the CNCM under number 1-464 or an isolated strain having the same characteristics as IP174178. The disclosure also relates to a pharmaceutical composition or a nutraceutical composition including such a strain, and to the use thereof for the treatment or prevention of genital infections.

### CROSS-REFERENCE TO RELATED APPLICATIONS

This application is a National Phase Entry of International Application No. PCT/EP2013/064424, filed on Jul. 9, 2013, which claims priority to French Patent Application Serial No. 1256569, filed on Jul. 9, 2012, both of which are incorporated by reference herein.

### BACKGROUND

The present invention relates to a novel specific strain of Lactobacillus crispatus, isolated from samples of healthy tissue, and to any strain having the same characteristics, as well as to compositions including same.

In a healthy woman, the urogenital flora comprises nearly 50 different species of microorganisms. Among these microorganisms, 95% of the population is composed of various strains of lactobacilli, including “Döderlein's bacillus”. These lactobacilli help protect against pathogens by various mechanisms, including the production of hydrogen peroxide, lactic acid and bacteriocins, the inhibition of adhesion and the spreading of pathogens. In particular, these lactobacilli maintain an acidic pH by producing lactic acid from the glycogen present in the vaginal mucus. Thus, the growth of numerous pathogens of the vaginal flora, such as Gardnerella vaginalis, Prevotella bivia, Neisseria gonorrhoeae, mycoplasmas, Mobiluncus and, above all, Candida albicans, is inhibited.

The normal vaginal flora is thus principally composed of lactobacilli forming a protective biofilm on the surface of the mucous membrane. The lactobacilli most frequently observed in the vagina are notably Lactobacillus crispatus, Lactobacillus jensenii, Lactobacillus vaginalis, Lactobacillus iners and Lactobacillus gasseri.

Vulvovaginal candidiasis affects 70-75% of women at least once during their reproductive years, and about 40-50% will have a second episode. The incidence of recurrent vulvovaginal candidiasis (defined as at least 4 episodes per year, including 2 confirmed by mycological examination) has been estimated at 5-8%. This benign ailment has a very negative impact on patients' quality of life and generates significant healthcare expenditures. Such pathology is difficult to treat because of the multifactorial pathogenesis of this affection.

The traditional maintenance therapy using an oral or vaginal antifungal must be for at least 6 months, but the relapse rate remains high with 60-70% of women having a recurrence in the two months following discontinuation of the treatment. Moreover, antifungals have frequent side effects and their long-term use can promote an occurrence of bacterial vaginosis.

Bacterial vaginosis is due to a qualitative and quantitative imbalance of the normal vaginal flora which can lead to the virtually complete disappearance of lactobacilli, to the benefit of anaerobic flora, and also to the emergence of the bacteria such as Gardnerella vaginalis and Atopobium vaginae involved in bacterial vaginosis. Bacterial vaginosis is one of the most frequent vaginal infections with a frequency rate of 10 to 15. This benign pathology in women can be serious during pregnancy as it can cause premature deliveries, low birth-weights and spontaneous abortions.

Bacterial vaginosis and other imbalances of the vaginal microflora are commonly treated by antibiotic therapy. This treatment has the typical disadvantages of antibiotic treatments and is proving to be less and less effective. Furthermore, it aims to eliminate the pathogenic flora but also destroys the normal beneficial flora.

The oral or vaginal administration of beneficial lactobacilli has been described to promote vaginal health and to treat genital infections. In particular, the patent applications WO 84/04675, WO 2000/035465, WO 2006/045475, and U.S. Patent Publication No. 2002/0044926 describe the oral or vaginal administration of lactic bacteria to promote vaginal health and to prevent recurrences of vulvovaginal candidiasis. The preferred lactobacilli are Lactobacillus rhamnosus and Döderlein's bacillus (Lactobacillus acidophilus vaginalis). U.S. Pat. Nos. 6,093,394, 6,468,526 and 7,807,440, as well as U.S. Patent Publication No. 2010/0151026, describe the administration of specific Lactobacillus crispatus strains.

Among these strains, mention may be made of the following Lactobacillus crispatus strains:
The Lactobacillus crispatus strains deposited at the DSMZ (Germany) under the deposition numbers DSM 16735, 16736, 16738, 16739, 16740, 16741, 16742 and 16743, as described in the patent application EP 1 824 500; these strains are able to colonize the vaginal mucous membranes and thus to promote vaginal health; they also have good properties of adhesion to epithelial cells;
The Lactobacillus crispatus strains deposited at the CNCM (France) under the deposition numbers 1-3483, 1-3484 and 1-3486, as described in the patent application EP 1 812 023; these strains were selected for their ability to adhere to epithelial cells; they exhibit good resistance to the acidic pHs of the gastric environment, as well as a proven anti-pathogenic effect;
The Lactobacillus crispatus strain deposited at the BCCM/LMG (Belgium) under the deposition number P-20558 as described in the patent EP 1 427 808 B1; this strain is used in combination with other lactic strains, to be administered with the help of a tampon, this strain having the ability to be able to colonize the vaginal cavity, including during menstruation; and
the Lactobacillus crispatus strain deposited at the ATCC (USA) under the deposition number 202225, as described in the patent EP 1 011 721 B1; this strain exhibits good adhesion to epithelial cells, long viability, notably in the presence of the preservation matrix described in this document, as well as good production of lactic acid and H₂O₂.
The P-17631 strain of Lactobacillus crispatus as described in the patent application EP 949 330 is capable of fermenting glycogen and inhibiting the growth of Candida.

Most of these probiotic strains, isolated from vaginal samples from healthy women, are unable to survive in an acidic pH environment or to survive extended contact with bile. Vaginal pH is a parameter which varies significantly during a woman's life: physiological pH is 7 in prepubescent girls, drops to 3.8 in pubescent females, and is greater than 5.5 during menopause, absent hormone replacement therapy. These variations are related to multiple factors such as the presence of estrogen and colonization by lactobacilli.

It is therefore important for probiotic strains administered exogenously to be able to withstand the acidic physiological pHs of the vaginal cavity, and moreover to be able to withstand the pH of the gastric cavity during oral administration. Furthermore, a number of these strains are unable to use glycogen as a source of fermentable carbon (Martin R. et al., 2008). However, this property is advantageous because the vaginal mucous membrane commonly lacks glucose, and the production of lactic acid by the probiotic strain can be maintained only in the presence of a source of fermentable carbon.

### SUMMARY

The present invention relates to a novel isolated Lactobacillus crispatus strain, identified as IP174178, deposited at the CNCM on Jun. 22, 2012 under the accession number CNCM 1-4646, and to any L. crispatus strain having the same characteristics. In the present description below, any reference to Lactobacillus crispatus IP174178 will refer to this strain as well as to any L. crispatus strain having the same characteristics.

This novel strain IP174178 has the following structural characteristics:
it grows in aerobic-anaerobic conditions;
its rep-PCR profile is presented in FIG. 1;
the sequence of its 16S ribosomal DNA is presented in SEQ ID NO: 1. Its principal functional characteristics are:
its high rate of H₂O₂ and lactic acid production (more than 7 g/liter);
its ability to grow on glycogen as the only carbonaceous substrate;
its resistance to bile;
its resistance to acidic pHs (in particular to pHs between 2.5 and 4);
its ability to inhibit the development of pathogens of the urogenital region.

The strain Lactobacillus crispatus IP174178 has in particular a resistance to acidic pHs. More particularly, the strain Lactobacillus crispatus IP174178 has a resistance to bile and to acidic pHs. More particularly, the strain Lactobacillus crispatus IP174178 has a resistance to bile and to acidic pHs, and is capable of metabolizing glycogen.

The invention also relates to a pharmaceutical composition including at least 10⁹ CFU of this strain Lactobacillus crispatus IP174178, alone or in combination with other probiotic strains or other active ingredients. The invention also relates to the use of this strain IP174178 for the prevention and treatment of recurrent or non-recurrent genital and urogenital infections.

### DESCRIPTION OF THE FIGURES

FIG. 1: Graphical representation in the form of a dendrogram of the comparison of genotype profiles by rep-PCR.

FIG. 2: Graphical representation in the form of matrix of the similarity of the comparison of genotype profiles by rep-PCR.

FIG. 3: Test of adhesion of the strain IP174178 to vaginal epithelial cells.

### DETAILED DESCRIPTION

The present invention relates to a novel isolated Lactobacillus crispatus strain identified as IP174178 and deposited at the CNCM under the accession number 1-4646, and to any isolated L. crispatus strain having the same characteristics as this isolated strain. The term “lactobacilli” refers to all Lactobacillus bacteria, Gram-positive bacteria, immobile, of variable shapes and sizes, and facultative anaerobes. Most lactobacilli convert lactose and other simple sugars into lactic acid. They are found in the vagina and the gastrointestinal tract and they constitute an important component of the intestinal and vaginal flora.

Lactobacillus crispatus is one of the predominant species of the vaginal flora in Europe, the United States and Japan (Antonio, MAD et al., 1999). The isolated strain Lactobacillus crispatus IP174178 has the following functional characteristics:
high production of H₂O₂ and lactic acid (more than 7 g/liter);
ability to grow on glycogen, an endogenous substrate secreted by the vaginal mucous membrane;
resistance to acidic pHs and to bile;
ability to inhibit the development of pathogens;
adhesion to vaginal epithelial cells.

This ability to grow on glycogen is relatively rare, and is particularly advantageous for strains intended to develop in the genital tract, where glucose is often absent, and where the only source of carbon is glycogen. Lactic acid production by this strain is thus maintained even in the absence of glucose (see Table 5). The ability to withstand acidic pHs is a highly advantageous characteristic, because it allows the strain according to the invention, on the one hand, to resist gastric pH when administered orally, and, on the other hand, to reproduce in the female genital tract even in the case of abnormally acidic secretions. The ability to resist bile makes it possible to envisage oral administration of this strain, and not solely vaginal administration. The invention also relates to a pharmaceutical composition including the strain Lactobacillus crispatus IP174178.

In the composition according to the invention, the number of bacteria per gram of composition is preferably greater than 10⁸, and is notably between 10⁸ and 10¹⁰. According to a typical aspect of the invention, the composition includes at least 10⁹ CFU of the Lactobacillus crispatus strain. The term “CFU” refers to “colony forming unit” and is the unit of measure generally recognized by persons skilled in the art to quantify bacteria capable of founding a colony.

Preferably, the Lactobacillus crispatus strain is lyophilized. The strain can be the only lyophilized element of the composition, but preferably the strain is lyophilized in a medium including additional constituents added before or after the lyophilization step.

According to another typical aspect of the invention, the pharmaceutical composition further includes a preservation matrix and/or excipients well-known to persons skilled in the art, and optionally other active ingredients having a complementary action. According to a typical aspect of the invention, the composition is in powder form, which can be placed in a capsule, or which can be integrated into any suitable type of carrier such as a cream or a nutraceutical composition.

In particular, this composition can include the following active ingredients: hormones and/or anti-inflammatory agents and/or bactericidal agents and/or antifungal agents. Persons skilled in the art will be able to determine which active ingredients can be advantageously coupled with the strain Lactobacillus crispatus IP174178.

According to a preferred aspect of the invention, the composition includes sodium thiosulfate, preferably in a concentration of at least 100 mg/gram of powder including between 10⁸ and 10¹⁰ CFU of Lactobacillus crispatus IP174178. The examples below illustrate a composition called “Formulation A” which includes 113 g/l of sodium thiosulfate before lyophilization, and finally 230 mg of sodium thiosulfate/g of powder. The presence of sodium thiosulfate makes it possible to potentiate the anti-pathogenic effect of the strain IP174178 (see Table 6C). The composition according to the invention can also include other Lactobacillus strains.

According to the invention, the pharmaceutical composition can be formulated for vaginal administration or for oral administration. In particular, the bacteria intended to be administered orally were tested for their resistance to bile, their resistance to acidic pH and their ability to grow in the presence of bile. The inventive strain IP174178 has these characteristics enabling it to be administered orally.

For pharmaceutical compositions for oral administration, the suitable formulations are in particular tablets, capsules, powders, granules, solutions and oral suspensions. For pharmaceutical compositions for vaginal administration, the suitable formulations are in particular creams, capsules and vaginal suppositories and tampons. The composition according to the invention is in particular intended to be used in the treatment of genital and urogenital infections such as vaginosis and candidiasis.

The term “vaginosis” refers to an imbalance of the vagina's microbial flora. It is characterized by the disappearance of lactobacilli and the multiplication of pathogenic bacteria such as those listed above. The term “candidiasis” refers to a vaginal infection caused by yeasts of the genus Candida.

These infections are related to the presence of pathogens, in particular belonging to the family of Candida albicans, Gardnerella vaginalis, mycoplasmas or Mobiluncus. Candida albicans is the most important and best known yeast species of the genus Candida. It causes fungal infections (candidiasis, also called candidosis) mainly of the digestive and genital mucous membranes. Gardnerella vaginalis is a bacterium with a shape of a pleomorphic rod or coccobacillus. It is a bacterium frequently found in cases of vaginosis (nonspecific vaginitis), either as the only pathogenic bacterium or in combination with other bacteria. It produces a perforating toxin that only affects human cells.

The term “mycoplasmas” refers to a family of more than 100 species of bacteria that are insensitive to the families of antibiotics that target cell walls (penicillin or beta-lactams). In particular, Mycoplasma genitalium is responsible for genital and urogenital infections (urethritis, cervicitis, vaginitis, salpingitis) and sterility problems. Mobiluncus is an anaerobic Gram-positive bacterium often found associated with Gardnerella vaginalis in bacterial vaginosis.

The invention also relates to a nutraceutical composition including the strain Lactobacillus crispatus IP174178, or a strain having similar characteristics. Such a nutraceutical composition will be provided in the form of a food composition or food supplement. A food composition will be composed of the strain Lactobacillus crispatus IP174178, preferably lyophilized, mixed with food products such as dairy products, fats, products containing cereals, confectioneries or beverages. The food supplement can in particular be provided in the form of capsules or soft capsules of gelatin or plant matter, in the context of the present invention.

The invention also relates to a medical device including one of the compositions as described above, this medical device being in particular a vaginal tampon, spatula, sanitary napkin, genital soap, etc. The invention also relates to the use of the isolated strain Lactobacillus crispatus IP174178, for the prevention and treatment of genital and urogenital infections such as vaginosis and candidiasis.

The optimal modes of administration, dosing schedules and dosage forms of the compositions according to the invention can be determined according to the criteria generally taken into account in the establishment of a pharmaceutical treatment suited to a patient, such as, for example, the patient's age or weight, the gravity of the patient's general state, the tolerance for the treatment and the side effects observed. The isolated strain Lactobacillus crispatus IP174178 will be used both in local (vaginal) administration and in oral administration, both types of administration being envisaged alone or in combination.

According to another aspect of the invention, the isolated strain Lactobacillus crispatus IP174178 is used to maintain a beneficial vaginal flora in a woman without an infection. According to still another aspect of the invention, the isolated strain Lactobacillus crispatus IP174178 is used to restore a beneficial vaginal flora following an antibiotic treatment. The invention thus relates to a pharmaceutical composition comprising the isolated strain Lactobacillus crispatus IP174178, for use in the maintenance or restoration of a beneficial vaginal flora.

### EXAMPLES

### Example 1

### Isolation and Selection of the Strain L. crispatus IP174178

Seven tests were applied to 10 strains isolated from healthy women, and preselected for their ability to produce hydrogen peroxide (H₂O₂). These tests are: growth in modified MRS medium (Man JC et al., 1960), ability to produce H₂O₂, ability to produce lactic acid, adhesion to cells of a vaginal cell line, ability to inhibit certain genital pathogens (disk inhibition method) and metronidazole resistance. Following these first tests, three strains identified as being of particular interest, in particular for their high production of H₂O₂ and lactic acid, were the subjects of growth tests in the presence of glycogen, the strain IP174178 only having the ability to use glycogen as a carbon source, which is an endogenous substrate secreted by the vaginal mucous membrane. In addition, this strain exhibits the best growth in the presence of bile. The results obtained are as follows:

TABLE 1
Last selection test of the strain IP174178

| | Rate of adhesion | Growth in | Growth in |
|---|---|---|---|
| Reference | to epithelial | the presence | the presence |
| strain | cells | of glycogen | of bile |
| PB 0040 | 37% | − | + |
| IP 174178 | 58% | ++ | ++ |
| PB 0042 | 62% | − | + |

### Example 2

### Biochemical Profile

The biochemical profiles are obtained from the VITEK II Compact system (bioMérieux), which is an automated system for identifying bacteria by comparing abilities to ferment specific sugars. We obtain the following biochemical profile: 11033303414010.

TABLE 2
Biochemical profile of the strain IP174178
Biochemical breakdown

| | | | | | | | | | | | | | | | | | |
|---|---|---|---|---|---|---|---|---|---|---|---|---|---|---|---|---|---|
| 2 | APPA | + | 4 | dGAL | − | 5 | ODC | − | 6 | PheA | + | 7 | ARG | − | 8 | PVATE | − |
| 9 | BGAL | − | 10 | PYRA | − | 11 | SUCT | − | 12 | TyrA | + | 13 | dGLU | + | 17 | BGLU | − |
| 18 | dMAL | + | 19 | dMAN | + | 21 | BXYL | − | 22 | O/129 R | + | 23 | ProA | + | 26 | LIP | − |
| 27 | AMAN | − | 30 | dMLZ | − | 31 | URE | − | 33 | SAC | + | 35 | dTRE | + | 36 | CIT | − |
| 37 | BGURI | − | 40 | ILATk | − | 41 | AGLU | + | 43 | dSOR | + | 44 | AGAL | − | 46 | GlyA | − |
| 47 | dMLT | − | 50 | dRIB | − | 51 | MTE | + | 52 | IGLM | − | 53 | OPS | (+) | 54 | BdFUC | − |
| 58 | CMT | + | 59 | 2KG | − | 61 | ESC | − | 62 | ELLM | − | 64 | dXYL | − | | | |

### Example 3

### Genotype Profile

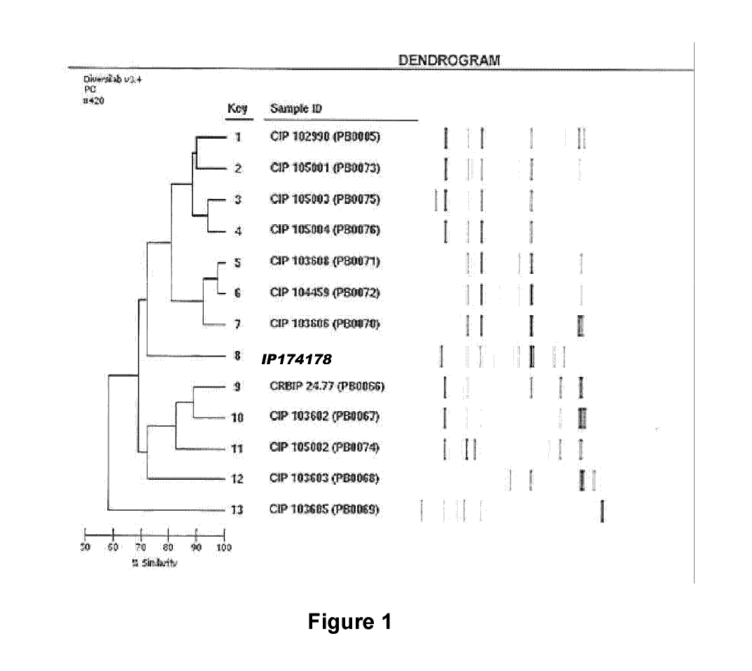

The genotype profile of the strain IP174178 was established on a DiversiLab system (bioMérieux). It corresponds to the amplification of DNA within the non-coding repeat sequences of the genome. The arrangement and size of these fragments are unique for each strain and thus make it possible to establish unique profiles for each (see FIG. 1).

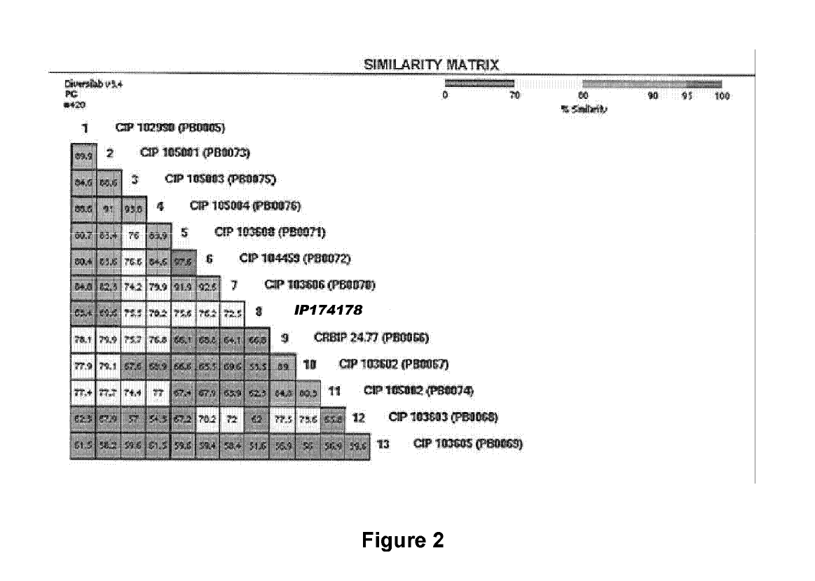

We then compared this rep-PCR profile with that of 26 Lactobacillus crispatus strains, including 11 strains preserved at the Institut Pasteur in order to verify the unique character of the strain (see FIG. 2). In each case the strain IP174178 comprised many differences, proof of its uniqueness. Moreover, the strain IP174178 is able to grow on glycogen alone, which has an advantage for its implantation in the vaginal mucous membrane, and has a high rate of production of H₂O₂ and lactic acid.

### Example 4

### Sequencing and Identification of Specific Oligonucleotides

Sequencing of the strain IP174178 was carried out using the Roche 454 technique. Following this work, an annotation was carried out. In the end, 1982 genes coding for proteins were identified.

TABLE 3
Genes annotated in the strain IP174178

| Name of the | Known | Hypothetical proteins | | |
|---|---|---|---|---|
| annotated strains | proteins | nonspecific | specific | Total |
| Lactobacillus crispatus | 1439 | 75 | 468 | 1982 |

The sequencing operation made it possible to undertake complementary molecular biology work on the strain in order to define specific PCR primer pairs. In the end, three PCR primers were selected and are specific to the strain during an amplification step consisting of 30 cycles. These primers make it possible to easily and very specifically identify the IP174178 strain of L. crispatus in a biological sample, for example.

TABLE 4
Sequences of the specific PCR primers for the
strain IP174178

| | Sequence | | |
|---|---|---|---|
| Name | number | Sequence | Direction |
| Contig00070 | SEQ ID NO: 2 | CAACAGGCAT | sense |
| | | CCCTAAGTCT | |
| | SEQ ID NO: 3 | CAACGTCTTC | antisense |
| | | AGACCACATC | |
| Contig00074 | SEQ ID NO: 4 | TCTGCCTCAT | sense |
| | | GTAGATCCAA | |
| | SEQ ID NO: 5 | GCTCCATCTT | antisense |
| | | TAAGACCCAA | |
| Contig00145 | SEQ ID NO: 6 | AAGTGTTATG | sense |
| | | GGCCTAGTCG | |
| | SEQ ID NO: 7 | GCCACCACTT | antisense |
| | | TTGCTTTAAT | |

### Example 5

### Preparation of “Formulation A”

The strain IP174178 is cultivated in the following culture medium: 116 g/l milk, 15 g/l dextrose, 10 g/l autolyzed yeast, 2 ml/l Tween.

After inoculation of the culture medium, fermentation is maintained at a temperature of 37° C. by a suitable thermostatically controlled device for 48 to 72 hours. At the conclusion of fermentation, the culture, having reached a count greater than or equal to 10⁸ CFU/ml, is poured into a mixing tank. The culture is shaken rapidly to break the curd and the following lyophilization adjuvants are added: 110 g/l milk, 101.5 g/l FOS, 9.5 ml/l sodium glutamate, 5.25 g/l ascorbic acid, 113 g/l sodium thiosulfate.

Lyophilization is then carried out according to the standard conditions well-known to persons skilled in the art: the bacteria are distributed aseptically on stainless steel trays, frozen quickly at −40° C., and then subjected to a sublimation operation at −22° C. Incubation is then carried out at 37° C. The resulting product then is crushed and filtered under controlled atmosphere. The powder obtained can advantageously be mixed with 1% of magnesium stearate in order to facilitate the filling of capsules. The powder thus obtained is distributed in “0” size capsules in an amount of 350 mg of powder per capsule.

### Example 6

### Functional Characteristics of the Strain IP174178 and of ‘Formulation A’

The strain identified as IP174178 was tested in parallel, alone and in its ‘Formulation A’ as presented in example 5, to determine its functional characteristics. In the examples below, the expression ‘pure strain’ or ‘strain alone’ or ‘strain IP174178’ refers to the strain IP174178 with no adjuvant, and notably without the compounds of Formulation A, resulting from colonies isolated on agar after preservation in cryogenic vials in a standard preservation medium.

### Antimicrobial Activities Demonstration of Hydrogen Peroxide Production

The lactic bacteria are cultured in MRS agar medium supplemented with vitamin B12, peroxidase and tetramethylbenzidine. After incubation for 48 hours in anaerobiosis, the culture dishes are placed in contact with ambient oxygen and the colonies producing hydrogen peroxide quickly turn blue. An arbitrary scale from 0 to 5 is used to note the intensity of H₂O₂ production.

TABLE 5
Functional characteristics of the strain IP174178

| | H₂O₂ | Lactic acid production (g/l) | |
|---|---|---|---|
| | production | Glucose source | Glycogen source |
| Formulation A | 5 | D-LACT: 3.69 | D-LACT: 3.91 |
| | | L-LACT: 4.88 | L-LACT: 4.74 |
| Strain IP174178 | 5 | D-LACT: 5.90 | D-LACT: 5.65 |
| | | L-LACT: 3.10 | L-LACT: 3.16 |

The results reported above illustrate that:
The production of hydrogen peroxide is maximum according to the arbitrary scale used;
The strain uses in a virtually equivalent manner, with glucose as well as with glycogen, the metabolic pathways enabling the synthesis of D- and L-lactates. Because glycogen is one of the principal sources of carbon in the vaginal environment, this strain possesses the properties required to produce lactic acid in vivo.

### Inhibition of the Growth of C. albicans and G. vaginalis

Objective: Evaluate the ability of the Lactobacillus strains to inhibit the growth of a pathogenic strain: Candida albicans; the same protocol is applied for G. vaginalis, adapted to this pathogen.

### Description of the Protocol

Measure the growth of the pathogens alone and in the presence of a culture of a Lactobacillus strain with determination of titer from T0 to T28h. Culture media used for preparing inocula and for reading titers:
Sabouraud broth and Sabouraud agar for Candida albicans (at 37° C.)
MRS broth/agar for the Lactobacillus strains.
   The culture broth used in the test is a homogeneous mixture of the culture medium of the pathogen and the culture medium of the probiotic.

### Preparation of the Test

Preparation of inocula:
Candida albicans: culture on Sabouraud broth—0.3 ml in 30 ml at 37° C. for 48 hours.
Probiotic strain:
   Formulation A: 0.35 g of powder of Formulation A is placed in 10 ml of MRS broth—in an incubator at 37° C. for 48 hours.
   Pure strain IP174178: 0.2 ml of pure strain, resulting from colonies isolated on agar, preserved beforehand in a cryogenic vial in a preservation medium, is placed in 10 ml of MRS broth—in an incubator at 37° C. for 48 hours.

Preparation of the control Candida:

To produce the control Candida, 5 ml of Sabouraud broth inoculated with the pathogenic strain (titer near 1·10⁸ CFU/ml) is placed with 5 ml of non-inoculated MRS broth.

Placing the inocula in contact:

Place 5 ml of the pathogenic strain with 5 ml of the strain according to the invention in these various configurations.

Samples are taken and titers are determined at T0, T4h, T24h and T28h. The results are expressed in CFU/ml. The concentration in CFU at TO is of course not the same in the three cases compared (due to working conditions) but the values at T0 are of the same order of magnitude, and thus the difference has no effect on the result.

TABLE 6A
Inhibition of Candida albicans

| | Contact time in hours | | | |
|---|---|---|---|---|
| | 0 | T4 h | T24 h | T28 h |
| Pure strain IP174178 | 1.24 · 10⁷ | 7.87 · 10⁶ | 1.40 · 10⁶ | 4.60 · 10⁵ |
| Formulation A | 5.43 · 10⁶ | 5.47 · 10⁶ | 0 | 0 |
| Control | 6.20 · 10⁶ | 9.72 · 10⁶ | 1.72 · 10⁷ | 1.40 · 10⁷ |
| Candida albicans | | | | |

TABLE 6B
Inhibition of Gardnerella vaginalis

| | Contact time in hours | | | |
|---|---|---|---|---|
| | 0 | T4 h | T24 h | T28 h |
| Pure strain IP174178 | 1.53 · 10⁷ | 1.67 · 10⁶ | 0 | 0 |
| Formulation A | 3.87 · 10⁶ | 6.73 · 10¹ | 0 | 0 |
| Gardnerella vaginalis | 6.33 · 10⁷ | 3.83 · 10⁷ | 6.71 · 10⁷ | 5.05 · 10⁷ |
| control | | | | |

TABLE 6C
Inhibition of Candida albicans - influence of sodium thiosulfate
concentration in the formulation of the strain IP174178
A Formulation A1 and a Formulation A2 were prepared by proceeding as
for Formulation A, except that Formulation A1 does not contain sodium
thiosulfate and Formulation A2 contains only 50 g/l of sodium
thiosulfate. The inhibition of C. albicans growth was measured in the
three cases.

| | Reduction in log₁₀ | | |
|---|---|---|---|
| | 4 h | 24 h | 28 h |
| Formulation A1 (0 g/l thiosulfate) | 0 | 1 | 2 |
| Formulation A2 (50 g/l thiosulfate) | 0 | 3 | 3 |
| Formulation A (113 g/l thiosulfate) | 0 | 6 | 7 |

In this table, the results are expressed as the decimal logarithmic (log₁₀) reduction of the number of CFU between the count at T0 and the various sampling points (T4h, T24h and T28h).

The results presented above, in Tables 6A-6C, indicate that:
The inhibition of the growth of the pathogenic C. albicans is real as of 24 hours, whether by the pure strain or by the strain in Formulation A. However, only Formulation A makes it possible to achieve complete inhibition after 24 hours.
The inhibition of the growth of the pathogenic G. vaginalis is complete after 24 hours of co-culture, in the presence of the strain alone or in its Formulation A; the inhibition of the growth of the pathogen is nevertheless faster with the strain in its Formulation A (after 4 hours).
The presence of sodium thiosulfate in Formulation A makes it possible to potentiate the anti-pathogenic effect of the strain IP174178. Indeed, the greater the concentration of sodium thiosulfate, the greater the inhibition observed at 24 hours.

### Gastric Resistance

According to the individual and especially to the time of day, gastric pH varies between 2.5 and 5. The resistance of the strain IP174178, alone or in Formulation A, was tested in media at pH=2.5, 3 and 4. The results are expressed as the reduction in the quantity of bacteria, over time.

TABLE 7
Resistance of the strain alone or in its Formulation A, at low pH

| | Resistance at pH 2.5 | | Resistance at pH 3 | | Resistance at pH 4 | |
|---|---|---|---|---|---|---|
| | Reduction (log₁₀) | | Reduction (log₁₀) | | Reduction (log₁₀) | |
| | 45 min | 90 min | 45 min | 90 min | 45 min | 90 min |
| Formulation A | −1.12 log | −4.36 log | −0.18 log | −0.72 log | −0.02 log | −0.15 log |
| Strain IP174178 | −0.73 log | −2.59 log | −0.51 log | −0.58 log | −0.68 log | −0.80 log |

The results show that after 90 minutes, at pH=3 or 4, the loss of bacteria is small, less than one log₁₀. The resistance at pH=2.5 is lower, notably when the strain is used in its Formulation A.

Resistance to bile was also tested, over time (60, 120, 180 and 240 minutes), in the presence of 3% bile. The results are expressed by comparing the quantity of surviving bacteria in the bile-free control medium and in the medium containing 3% bile.

TABLE 8
Resistance of the strain alone or in its
Formulation A, in the presence of bile

| | Comparison of medium without bile/ | | | | |
|---|---|---|---|---|---|
| Presence of 3% bile | medium with bile (log₁₀) | | | | |
| in the culture medium | 0 | 60 min | 120 min | 180 min | 240 min |
| Pure strain IP174178 | −0.46 | −0.50 | −0.54 | −0.63 | −0.86 |
| Formulation A | −0.56 | −0.66 | −0.58 | −0.68 | −0.79 |

After 240 minutes (4 hours), the difference between the control cultivated without bile and the medium with bile is less than one decimal logarithm, which means that the growth of the bacterium IP174178 is affected little by the presence of bile.

### Resistance to Antibiotics

TABLE 9
Resistance to antibiotics of the strain alone or in its Formulation A

| | Formulation | Pure strain |
|---|---|---|
| Antibiotics tested | A | IP174178 |
| Tetracycline | Sensitive | Sensitive |
| Colistin | Resistant | Resistant |
| Fusidic acid | Resistant | Resistant |
| Chloramphenicol | Sensitive | Sensitive |
| Streptomycin | Resistant | Resistant |
| Trimethoprim | Resistant | Resistant |
| sulfamethoxazole | | |
| Metronidazole 50 μg | Resistant | Resistant |
| Nalidixic acid | Resistant | Resistant |
| Erythromycin | Sensitive | Sensitive |
| Polymyxin | Resistant | Resistant |
| Pipemidic acid | Resistant | Resistant |
| Clindamycin 25 μg | Sensitive | Sensitive |
| Clindamycin 2 μg | Sensitive | Sensitive |

The following genes, known to be involved in resistance to the antibiotics listed in Table 10 below, were identified as being expressed in the L. crispatus strain:

Antibiotics

| Family | Name | L. crispatus IP174178 |
|---|---|---|
| Aminoglycoside | Gentamycin | aac3 |
| | Neomycin | |
| | Kanamycin | |
| | Streptomycin | |
| Diaminopyrimidine | Trimethoprim | dfrA |
| Fusidanine | Fusidic acid | fusA |
| Quinolone | Nalidixic acid | gyrA, norA |
| Fluoroquinolone | Ciprofloxacin | gyrA, norA |

### Adhesion to Vaginal Epithelial Cells CRL-2616

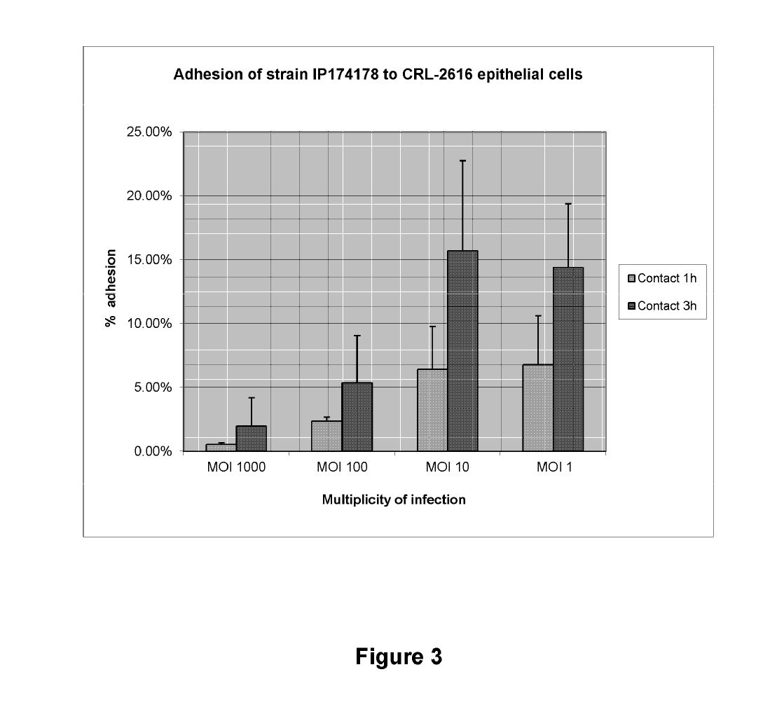

The results are presented in FIG. 3, and relate only to the strain IP174178 alone. Over time, the percentage of adhesion of IP174178 bacteria to CRL-2616 cells increases and reaches 15% adhesion after 3 hours, when the multiplicity of infection is 10 or 1.

### REFERENCES

### Patent Documents

WO 84/04675

WO 2000/035465

WO 2006/045475

US 2002/0044926

U.S. Pat. No. 6,093,394

U.S. Pat. No. 6,468,526

U.S. Pat. No. 7,807,440

US 201 0/01 51 026

EP 0 949 330

EP 1 824 500

EP 1 812 023

EP 1 427 808

EP 1 011 721

### SCIENTIFIC LITERATURE

Martin R, SoberóN, Vaneechoutte M, Flórez AB, Vázquez F, Suárez J E. Characterization of indigenous vaginal lactobacilli from healthy women as probiotic candidates. Int Microbiol. 2008 December; 11(4):261-6.

Antonio, M A D, S E Hawes, and S L Hillier. The identification of vaginal Lactobacillus species and the demographic and microbiologic characteristics of women colonized by these species. J. Infect. Dis. (1999) 180: 1950-1956.

Man J. C., de Rogosa M. and Sharpe, M E. A medium for the cultivation of Lactobacilli, Appl. Bact. (1960) 23, 130-135.

1. An isolated strain of Lactobacillus crispatus, identified as IP174178 and deposited at the CNCM under the accession number I-4646.

2. A pharmaceutical composition including a strain according to claim 1.

3. The pharmaceutical composition according to claim 2, including at least 10⁹ CFU of said strain.

4. The pharmaceutical composition according to claim 2, further including a preservation matrix.

5. The pharmaceutical composition according to claim 2, further including sodium thiosulfate.

6. The pharmaceutical composition according to claim 2, formulated for vaginal administration or for oral administration.

7. (canceled)

8. A medical device, in particular a vaginal tampon, containing a composition according to claim 2.

9. A nutraceutical composition including a strain according to claim 1.

10. (canceled)

11. The pharmaceutical composition according to claim 2, further including other active ingredients.

12. A method the treatment and prevention of genital and urogenital infections comprising administering to a patient in need thereof the pharmaceutical composition according to claim 2.

13. The method of claim 12, wherein the infection is vaginosis or candidiasis.

14. A method for maintaining or restoring beneficial vaginal flora comprising administering to a patient in need thereof the pharmaceutical composition according to claim 2.
